(19) United States
(12) Patent Application Publication
(10) Pub. No.: US 2004/0105906 A1
(43) Pub. Date: Jun. 3, 2004

(54) Antiagionecic, antitumor, chemopreventative agents

(76) Inventors: Jack L. Arbiser, Atlanta, GA (US); Xianhe Bai, Tucker, GA (US)

Correspondence Address: Christopher B Linder, Thomas Kayden Horstemeyer & Risley, 100 Galleria Parkway NW, Atlanta, GA, 30339-5948, US

(21) Appl. No.: 10/472,512

(22) PCT Filed: Mar. 22, 2002
(86) PCT No.: PCT/US02/08852

(51) Int. Cl. A61K 35/78

(52) U.S. Cl. 424/770

(57) ABSTRACT

Pharmaceutical compositions and methods of treating conditions such as angiogenic-, neoplastic-, and cancer-related conditions and skin conditions are disclosed. The methods include administering to a host in need of treatment an effective amount of at least one honokiol-type compound and/or at least one magnolol-type compound. In addition, the pharmaceutical compositions include at least one honokiol-type compound and/or at least one magnolol-type compound in combination with a pharmaceutically acceptable carrier. The honokiol-type compound and/or magnolol-type compound are present in a dosage level effective to treat conditions listed above.

### CROSS-REFERENCE TO RELATED APPLICATION

This application claims priority to copending U.S. provisional application entitled, “Antiangiogenic, Antitumor, and Chemopreventative Agents, Including Magnolol and Honokiol Derived From Magnolia Magniflora,” having ser. No. 60/278,149, filed Mar. 23, 2001, which is entirely incorporated herein by reference.

### TECHNICAL FIELD

The present invention is generally related to compositions and methods for administration of the compositions to hosts and, more particularly, is related to a compositions designed for treatment of cancer related conditions and skin conditions and methods of administration thereof.

### BACKGROUND

Cancer can be defined as an abnormal growth of tissue characterized by a loss of cellular differentiation. This term encompasses a large group of diseases in which there is an invasive spread of such undifferentiated cells from a primary site to other parts of the body where further undifferentiated cellular replication occurs, which eventually interferes with the normal functioning of tissues and organs.

Cancer can be defined by four characteristics which differentiate neoplastic cells from normal ones: (1) clonality—cancer starts from genetic changes in a single cell which multiplies to form a clone of neoplastic cells; (2) autonomy—biochemical and physical factors that normally regulate cell growth, do not do so in the case of neoplastic cells; (3) anaplasia—neoplastic cells lack normal differentiation which occurs in nonmalignant cells of that tissue type; (4) metastasis—neoplastic cells grow in an unregulated fashion and spread to other parts of the body.

Each cancer is characterized by the site, nature, and clinical cause of undifferentiated cellular proliferation. The underlying mechanism for the initiation of cancer is not completely understood; however, about 80% of cancers may be triggered by external stimuli such as exposure to certain chemicals, tobacco smoke, ultra violet rays, ionizing radiation, and viruses. Development of cancer in immunosuppressed individuals indicates that the immune system is an important factor controlling the replication and spread of cancerous cells throughout the body.

The high incidence of cancer in certain families, though, suggests a genetic disposition towards development of cancer. The molecular mechanisms involved in such genetic dispositions fall into a number of classes including those that involve oncogenes and suppressor genes.

Proto-oncogenes are genes that code for growth promoting factors necessary for normal cellular replication. Due to mutation, such proto-oncogenes are inappropriately expressed—and are then termed oncogenes. Oncogenes can be involved in malignant transformation of the cell by stimulating uncontrolled multiplication.

Suppressor genes normally act by controlling cellular proliferation through a number of mechanisms including binding transcription factors important to this process. Mutations or deletions in such genes contribute to malignant transformation of a cell.

Malignant transformation develops and cancer results because cells of a single lineage accumulate defects in certain genes such as proto-oncogenes and suppressor genes responsible for regulating cellular proliferation. A number of such specific mutations and/or deletions must occur in a given cell for initiation of uncontrolled replication. It is believed that genetic predisposition to a certain type of cancer results from inheritance of genes that already have a number of mutations in such key regulatory genes and subsequent exposure to environmental carcinogens causes enough additional key mutations or deletions in these genes in a given cell to result in malignant transformation. Changes in other types of genes could further the ability of tumors to grow, invade local tissue, and establish metastases at distant body sites.

Current treatments of cancer and related diseases have limited effectiveness and numerous serious unintended side effects. Cancer therapy is currently divided into five subspecialties: (1) surgery, (2) radiation therapy, (3) chemotherapy, (4) immunotherapy, and (5) antiangiogenic therapy. These treatments have progressed only incrementally during more than thirty years of intensive research to discover the origins of cancer and devise improved therapies for cancer and related diseases.

Current research strategies emphasize the search for effective therapeutic modes with less risk, including the use of natural products and biological agents. This change in emphasis has been stimulated by the fact that many of the consequences, to both patients and their offspring, of conventional cancer treatment result from their actions on genetic material and mechanisms. Efforts continue to discover both the origins of cancer at the genetic level and correspondingly new treatments, but such interventions also may have serious unanticipated effects.

The observation by Folkman that tumors are highly vascular, and the elucidation by him and others of a process termed “angiogenesis” through which many tumors derive a blood supply by the generation of microvessels, provided an important new avenue to the therapy of cancer and other diseases and disorders. Folkman, Proc. Natl. Acad. Sci. U.S.A. 95(16): 9064-6 (1998); C. R. Acad. Sci. III 316(9):909-918 (1993). Angiogenesis has now been recognized in inflammatory lesions and benign tumors, in addition to malignant tumors.

Mammals are characterized by complex cardiovascular systems that enable their warm-blooded nature, internal embryonic and fetal development, and successful population of extreme habitats. The development of an extensive capillary system, specialized in each organ and tissue, is an essential feature of mammalian cardiovascular system that provides optimal distribution of nutrients and other substances including hormones and defensive agents. The metabolic and physiologic needs of mammalian cells are met by their proximity to capillaries, and limited resources may be diverted by imbalance of this supply system.

Angiogenesis results primarily from the development of new or lengthened capillaries, and larger microvessels. Capillaries are formed primarily of specialized endothelial cells and the connective tissue layer to which they adhere, the basement membrane. The proliferation of endothelial cells and their migration and orientation to form capillaries is recognized as the key process regulated in the control of angiogenesis. Neovascularization is a form of angiogenesis marked by formation of blood vessels in a tissue or region previously devoid of blood vessel supply, for example the cornea of the eye. The mechanisms involved in angiogenesis are quite complicated, however, and no single one appears to be the sole controlling mechanism.

Mammals have effective mechanisms to regulate this vital process. Stimulation of angiogenesis in adult mammals, other than as a part of normal tissue repair, pregnancy or the menstrual cycle, is abnormal and often pathological. Many malignant tumors, benign tumors, and inflammatory lesions have the ability to evade or mobilize these regulatory mechanisms to support their growth and further malignant progression.

Development of effective preventive and treatment means has been hampered by inadequate understanding of the factors controlling this process. The premise of therapeutic development for such conditions is that effective treatment does not require destruction of the cells or tissues of origin. Reduction or prevention of the increased blood supply can be sufficient to prevent their growth, and the manifestation of the condition as a disease or pathological disorder.

Thus, a heretofore unaddressed need exists in the industry to address at least the aforementioned deficiencies and/or inadequacies in regard to preventing and treating cancer and related diseases.

### SUMMARY OF THE INVENTION

Briefly described, embodiments of the present invention include representative methods to treat conditions such as angiogenic-, neoplastic-, and cancer-related conditions and skin conditions. A representative method includes administering to a host in need of treatment an effective amount of at least one honokiol-type compound and/or at least one magnolol-type compound. Another method includes prophylactically treating one or more of the conditions listed above by administering to a host in need of treatment an effective amount of at least one honokiol-type compound and/or at least one magnolol-type compound.

Alternate embodiments of the present invention also include pharmaceutical compositions having at least one honokiol-type compound and/or at least one magnolol-type compound in combination with a pharmaceutically acceptable carrier. The at least one honokiol-type compound and/or at least one magnolol-type compound are present in a dosage level effective to treat conditions such as angiogenic-, neoplastic-, and cancer-related conditions and skin conditions.

Other systems, methods, features, and advantages of the present invention will be or will become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 is a diagram that illustrates honokiol-type compound and magnolol-type compound structures of the present invention.

FIG. 2 is a diagram that illustrates representative functional groups of the honokiol-type compound and magnolol-type compound structures shown in FIGS. 2, 3A, and 3B.

FIGS. 3A and 3B is a diagram that illustrates representative structures that are structurally similar to the honokiol-type compound and magnolol-type compound structures illustrated in FIG. 1.

FIG. 4 is a graph that illustrates the inhibition of SVR cell proliferation of honokiol-type and magnolol-type compounds.

### DETAILED DESCRIPTION

The present invention provides for compositions and methods of treating hosts having angiogenic-, neoplastic-, and cancer-related conditions and skin conditions. In addition, the present invention provides chemopreventative compositions and chemopreventative methods of treating hosts that are predisposed to angiogenic-, neoplastic-, and cancer-related conditions, as well as skin conditions.

As used herein, the term “host” includes both humans, mammals (e.g., cats, dogs, horses, etc.), and other living species that are in need of treatment. Hosts that are “predisposed to” angiogenic-, neoplastic-, and cancer-related conditions can be defined as hosts that do not exhibit overt symptoms of one or more of these conditions but that are genetically, physiologically, or otherwise at risk of developing one or more of these conditions. Thus, compositions of the present invention can be used prophylactically as chemopreventative agents for these conditions. Further, a “composition” can include one or more chemical compounds, as described below.

Embodiments of the present invention include compositions having at least one honokiol-type compound. Other embodiments of the present invention include compositions having at least one magnolol-type compound. Still other embodiments of the present invention include compositions having at least one honokiol-type and at least one magnolol-type compound. Further embodiments of the present invention include methods of treating conditions with compositions having at least one honokiol-type and/or at least one magnolol-type compound. Still further embodiments of the present invention include chemopreventative methods of treating conditions with compositions having at least one honokiol-type and/or at least one magnolol-type compound.

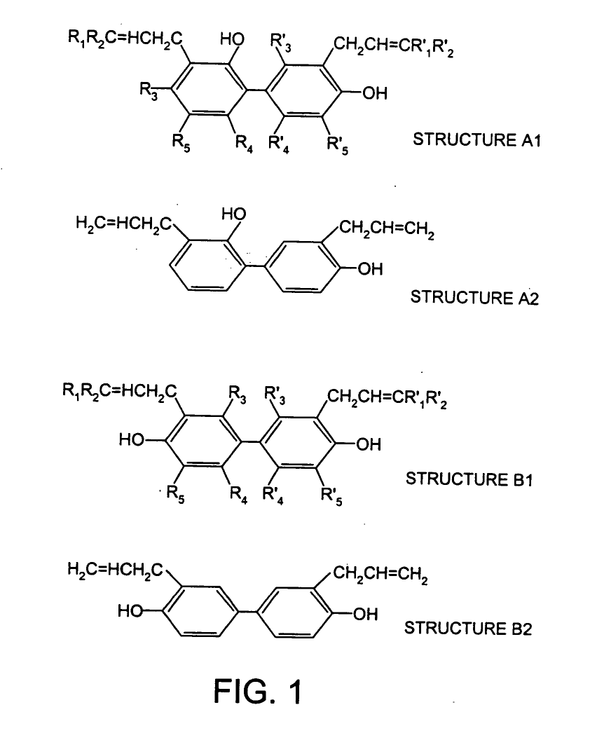

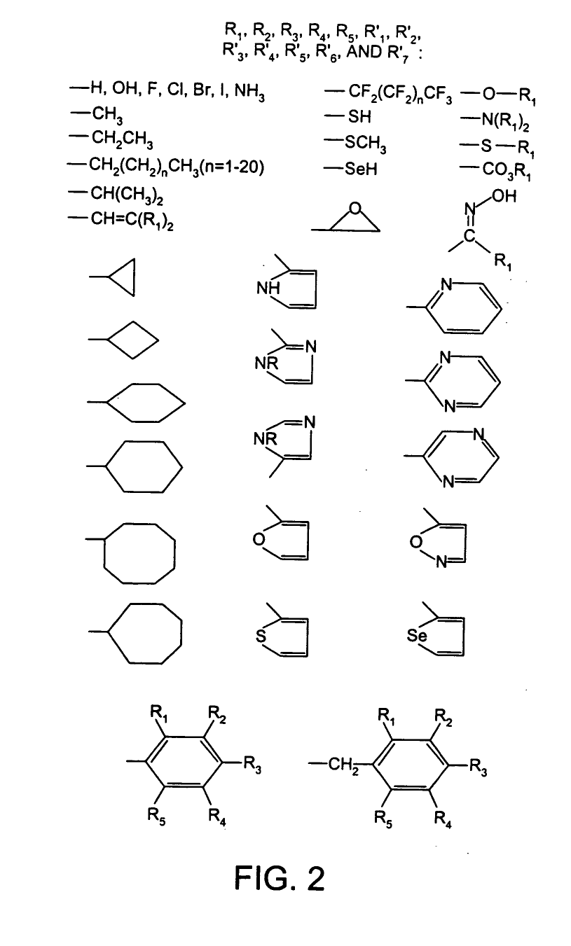

Honokiol-type compounds can include, but are not limited to, structure A1 illustrated in FIG. 1. More particularly, honokiol-type compounds can include structure A2 illustrated in FIG. 1. The functional groups of the honokiol-type compounds are indicated as R₁, R₂, R₃, R4, R₅, R′₁, R′₂, R′₃, R′₄, and R′₅. The functional groups include, but are not limited to, hydrogen, hydroxyl groups, amides, amines, hydrocarbons, halogenated hydrocarbons, cyclic hydrocarbons, cyclic heterocarbons, halogenated cyclic heterocarbons, benzyl compounds, halogenated benzyl compounds, organo selenium compounds, sulfide compounds, cabonyl compounds, thiol compounds, ether compounds, dinitrogen ring compounds, thiophene compounds, pyridine compounds, pyrrole compounds, imidazole compounds, and pyrimidine compounds. FIG. 2 is a diagram that illustrates exemplary functional groups of R₁, R₂, R₃, R4, R₅, R′₁, R′₂, R′₃, R′₄, and R′₅.

Where such forms exist, honokiol-type compounds may include honokiol-type compound analogues, homologues, isomers, or derivatives thereof, that function to treat angiogenic-, neoplastic-, and cancer-related conditions in a host, and/or function prophylactically as a chemopreventative composition. In addition, honokiol-type compounds can include pharmaceutically acceptable salts, esters, and prodrugs of the honokiol-type compounds described or referred to above.

Magnolol-type compounds include, but are not limited to, structure B1 illustrated in FIG. 1. More particularly, magnolol-type compounds can include structure B2 illustrated in FIG. 1. The functional groups of the magnolol-type compounds are indicated as R₁, R₂, R₃, R₄, R₅, R′₁, R′₂, R′₃, R′₄, and R′₅. The functional groups include, but are not limited to, hydrogen, hydroxyl groups, amides, amines, hydrocarbons, halogenated hydrocarbons, cyclic hydrocarbons, cyclic heterocarbons, halogenated cyclic heterocarbons, benzyl compounds, halogenated benzyl compounds, organo selenium compounds, sulfide compounds, cabonyl compounds, thiol compounds, ether compounds, dinitrogen ring compounds, thiophene compounds, pyridine compounds, pyrrole compounds, imidazole compounds, and pyrimidine compounds. FIG. 2 is a diagram that illustrates exemplary functional groups of R₁, R₂, R₃, R₄, R₅, R′₁, R′₂, R′₃, R′₄, and R′₅.

Where such forms exist, magnolol-type compounds can include magnolol-type compound analogues, homologues, isomers, or derivatives thereof, that function to treat angiogenic-, neoplastic-, and cancer-related conditions in a host and/or function prophylactically as a chemopreventative composition. In addition, magnolol-type compounds can include pharmaceutically acceptable salts, esters, and prodrugs of the magnolol-type compounds described or referred to above.

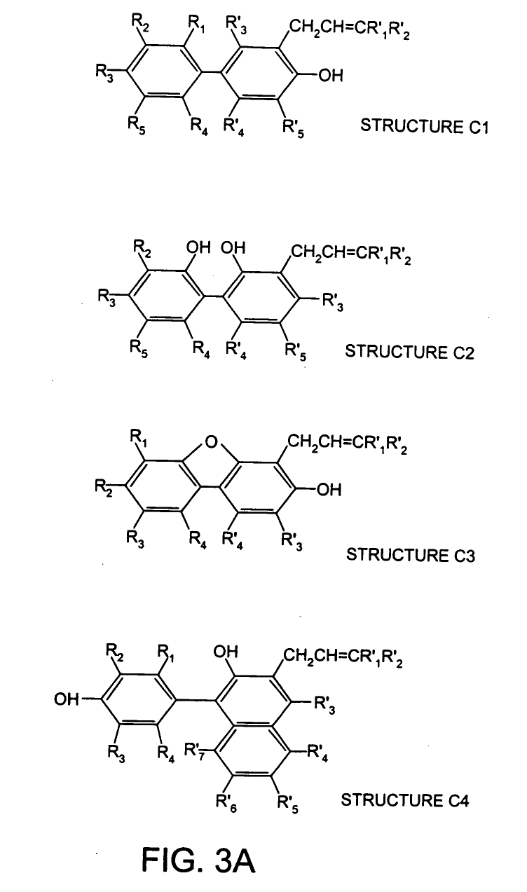

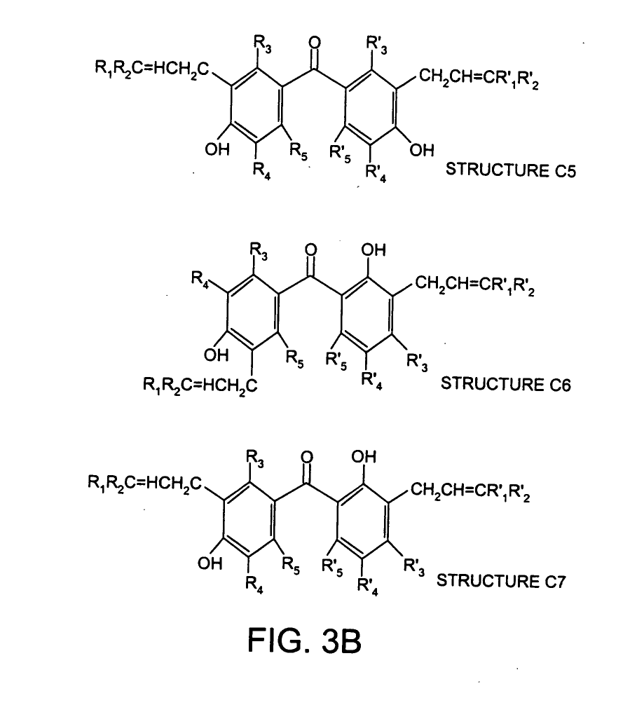

FIGS. 3A and 3B are diagrams that illustrate additional structures C1-C7 that are related to honokiol-type compounds and/or magnolol-type compounds. These compounds may be used instead of or in addition to the honokiol-type and/or magnolol-type compounds described or referred to above. In this regard, functional groups R′₆ and R′₇ can be any of the functional groups described or referred to above in FIG. 2.

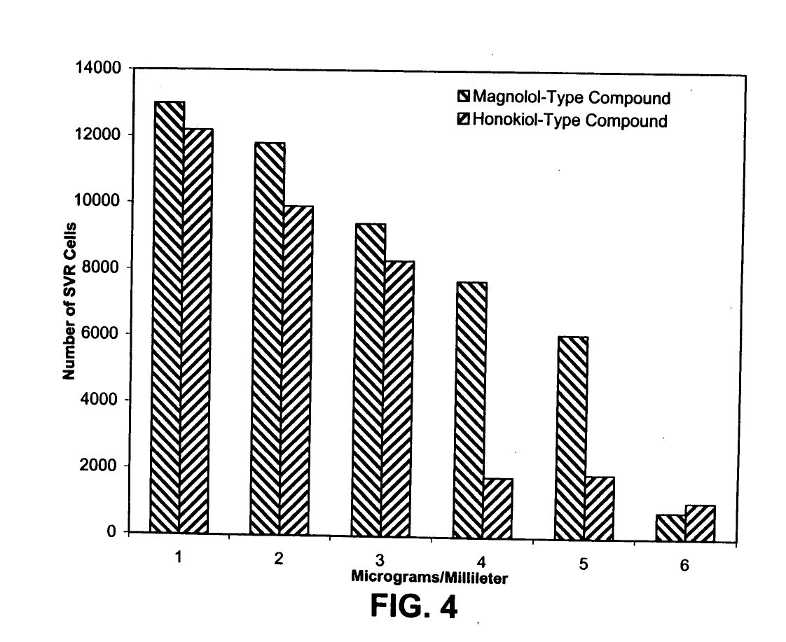

As illustrated in FIG. 4, honokiol-type compounds and magnolol-type compounds have been shown to be effective at decreasing the proliferation of SVR cells. In this regard, using inhibition of transformed SVR endothelial cells as a bioassay, honokiol-type compounds and magnolol-type compounds show enhanced activity in the SVR inhibition assay. Previously, bioassays of transformed SVR endothelial cells have been used to accurately predict in vivo responses to known angiogenesis inhibitors (Arbiser, et al. Proc. Natl. Acad. Sci, 94, 861-866 and Arbiser et al., J. Am. Acad. of Dermatol, 40, 925-929, which are herein incorporated by reference). Therefore, honokiol-type compounds and magnolol-type compounds may be used to inhibit angiogenesis, which is discussed in greater detail in Bai et al., Isolation. Characterization, And Antitumor Activity Of A Small Molecular Weight Compound From Magnolia Grandiflora, in press, which is herein incorporated by reference.

By “pharmaceutically acceptable salt” it is meant those salts which are, within the scope of sound medical judgement, suitable for use in contact with the tissues of hosts without undue toxicity, irritation, allergic response and the like, and are commensurate with a reasonable benefit/risk ratio and effective for their intended use. The salts can be prepared in situ during the final isolation and purification of one or more compounds of the composition, or separately by reacting the free base function with a suitable organic acid.

Representative acid addition salts include, but are not limited to, acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like.

Representative alkali or alkaline earth metal salts that may be used as the pharmaceutically acceptable salts include, but are not limited to, sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “pharmaceutically acceptable esters” as used herein refers to those esters of one or more compounds of the composition which are, within the scope of sound medical judgement, suitable for use in contact with the tissues of hosts without undue toxicity, irritation, allergic response, and the like, are commensurate with a reasonable benefit/risk ratio, and are effective for their intended use.

The term “pharmaceutically acceptable prodrugs” as used herein refers to those prodrugs of one or more compounds of the composition which are, within the scope of sound medical judgement, suitable for use in contact with the tissues of hosts without undue toxicity, irritation, allergic response, and the like, are commensurate with a reasonable benefit/risk ratio, and are effective for their intended use. Pharmaceutically acceptable prodrugs also include zwitterionic forms, where possible, of one or more compounds of the composition. The term “prodrug” refers to compounds that are rapidly transformed in vivo to yield the parent compound, for example by hydrolysis in blood. Compositions of this invention can be used to treat conditions such as, but not limited to, angiogenesis-related diseases, neoplastic-related diseases, cancer-related diseases, skin diseases, inflammatory disorders, coronary heart diseases, retinopathic diseases, etc. In addition, compositions of this invention can be used prophylactically as chemopreventative compositions that can be used to inhibit the development and/or slow the development of the conditions listed above.

Angiogenesis-related diseases include, but are not limited to, inflammatory, autoimmune, and infectous diseases; angiogenesis-dependent cancer, including, for example, solid tumors, blood born tumors such as leukemias, and tumor metastases; benign tumors, for example hemangiomas, acoustic neuromas, neurofibromas, trachomas, and pyogenic granulomas; rheumatoid arthritis; psoriasis; eczema; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; and wound granulation. In addition, compositions of this invention can be used to treat diseases such as, but not limited to, intestinal adhesions, atherosclerosis, scleroderma, warts, and hypertrophic scars (i.e., keloids). Compositions of this invention may also be useful in the treatment of diseases that have angiogenesis as a pathologic consequence such as cat scratch disease (Rochele minalia quintosa), ulcers (Helobacter pylori), tuberculosis, and leprosy.

Further, examples of cancers treatable by compositions of this invention include, but are not limited to, melanoma; high grade glioma, glioblastoma and other brain cancers; lung cancer; breast cancer; testicular cancer; gastro intestinal cancers, including colon, rectal, pancreatic, and gastric cancers; hepatocellular carcinoma; head and neck cancers; prostate cancer; carcinomas; renal cell carcinoma; adenocarcinoma; sarcomas; melanoma; hemangioendothelioma; lymphomas; leukemias; and mycosis fungoides.

Compositions of this invention can be used to treat these cancers and other cancers at any stage from the discovery of the cancer to advanced stages. In addition, compositions of this invention can be used in the treatment of the primary cancer and metastases thereof.

Furthermore, the skin diseases that may be treated with compositions of the invention include, but are not limited to, the malignant diseases angiosarcoma, hemangioendothelioma, basal cell carcinoma, squamous cell carcinoma, malignant melanoma and Kaposi's sarcoma, and the non-malignant diseases or conditions such as psoriasis, lymphangiogenesis, hemangioma of childhood, Sturge-Weber syndrome, verruca vulgaris, neurofibromatosis, tuberous sclerosis, pyogenic granulomas, recessive dystrophic epidermolysis bullosa, venous ulcers, acne, rosacea, eczema, molluscum contagious, seborrheic keratosis, and actinic keratosis.

Compositions of this invention may be suitable for oral, rectal, nasal, topical (including buccal and sublingual), vaginal, or parenteral (including subcutaneous, intramuscular, subcutaneous, intravenous, intradermal, intraocular, intratracheal, intracisternal, intraperitoneal, and epidural) administration.

The compositions may conveniently be presented in unit dosage form and may be prepared by conventional pharmaceutical techniques. Such techniques include the step of bringing into association one or more compositions of the present invention and one or more pharmaceutical carriers or excipients.

Compositions of the present invention suitable for oral administration may be presented as discrete units such as, but not limited to, tablets, caplets, pills or dragees capsules, or cachets, each containing a predetermined amount of one or more of the compositions; as a powder or granules; as a solution or a suspension in an aqueous liquid or a non-aqueous liquid; or as an oil-in-water liquid emulsion or a water-in-oil emulsion or as a bolus, etc.

Compositions of the present invention suitable for topical administration in the mouth include for example, lozenges, having the ingredients in a flavored basis, usually sucrose and acacia or tragacanth; pastilles, having one or more of the compositions of the present invention in an inert basis such as gelatin and glycerin, or sucrose and acacia; and mouthwashes, having one or more of the compositions of the present invention administered in a suitable liquid carrier.

Compositions of the present invention suitable for topical administration to the skin may be presented as ointments, creams, gels, and pastes, having one or more of the compositions administered in a pharmaceutical acceptable carrier.

Compositions of the present invention for rectal administration may be presented as a suppository with a suitable base comprising, for example, cocoa butter or a salicylate.

Compositions of the present invention suitable for nasal administration, when the carrier is a solid, include a coarse powder having a particle size, for example, in the range of 20 to 500 microns which is administered in the manner in which snuff is taken, (i.e., by rapid inhalation through the nasal passage from a container of the powder held close up to the nose). When the carrier is a liquid (for example, a nasal spray or as nasal drops), one or more of the compositions can be admixed in an aqueous or oily solution, and inhaled or sprayed into the nasal passage.

Compositions of the present invention suitable for vaginal administration may be presented as pessaries, tampons, creams, gels, pastes, foams or spray formulations containing one or more of the compositions and appropriate carriers.

Compositions of the present invention suitable for parenteral administration include aqueous and non-aqueous sterile injection solutions which may contain anti-oxidants, buffers, bacteriostats, and solutes which render the formulation isotonic with the blood of the intended recipient; and aqueous and non-aqueous sterile suspensions which may include suspending agents and thickening agents. The compositions may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules, and tablets of the kind previously described above.

Pharmaceutical organic or inorganic solid or liquid carrier media suitable for enteral or parenteral administration can be used to fabricate the compositions. Gelatin, lactose, starch, magnesium stearate, talc, vegetable and animal fats and oils, gum, polyalkylene glycol, water, or other known carriers may all be suitable as carrier media.

Compositions of the present invention may be used as the active ingredient in combination with one or more pharmaceutically acceptable carrier mediums and/or excipients. As used herein, “pharmaceutically acceptable carrier medium” includes any and all carriers, solvents, diluents, or other liquid vehicles, dispersion or suspension aids, surface active agents, isotonic agents, thickening or emulsifying agents, preservatives, solid binders, lubricants, adjuvants, vehicles, delivery systems, disintegrants, absorbents, preservatives, surfactants, colorants, flavorants, or sweeteners and the like, as suited to the particular dosage form desired.

Additionally, the compositions of the invention maybe combined with pharmaceutically acceptable excipients, and, optionally, sustained-release matrices, such as biodegradable polymers, to form therapeutic compositions. A “pharmaceutically acceptable excipient” refers to a non-toxic solid, semi-solid or liquid filler, diluent, encapsulating material or formulation auxiliary of any type.

Except insofar as any conventional carrier medium is incompatible with the compounds used in practicing embodiments of the invention, such as by producing any undesirable biological effect or otherwise interacting in a deleterious manner with one or more of the compounds of the pharmaceutical composition, its use is contemplated to be within the scope of the embodiments of this invention.

When used in the above or other treatments, a therapeutically effective amount of one or more of the components of the compositions may be employed in pure form or, where such forms exist, in pharmaceutically acceptable salt, ester, and prodrug form. By a “therapeutically effective amount” of one or more of the components of the composition it is meant a sufficient amount of one or more of the components to treat an angiogenic disease, (for example, to limit tumor growth, decrease tumor volume, or to slow or block tumor metastasis) at a reasonable benefit/risk ratio applicable to any medical treatment.

It will be understood, however, that the total daily usage of the compositions of the present invention will be decided by the attending physician within the scope of sound medical judgment. The specific therapeutically effective dose level for any particular host will depend upon a variety of factors, including for example, the disorder being treated and the severity of the disorder; activity of the specific composition employed; the specific composition employed, the age, body weight, general health, sex and diet of the patient; the time of administration; route of administration; rate of excretion of the specific compound employed; the duration of the treatment; drugs used in combination or coincidential with the specific composition employed; and like factors well known in the medical arts. For example, it is well within the skill of the art to start doses of the composition at levels lower than those required to achieve the desired therapeutic effect and to gradually increase the dosage until the desired effect is achieved.

Compositions of the present inventions are preferably formulated in dosage unit form for ease of administration and uniformity of dosage. “Dosage unit form” as used herein refers to a physically discrete unit of the composition appropriate for the host to be treated. Each dosage should contain the quantity of composition calculated to produce the desired therapeutic affect either as such, or in association with the selected pharmaceutical carrier medium.

Preferred unit dosage formulations are those containing a daily dose or unit, daily sub-dose, or an appropriate fraction thereof, of the administered ingredient. For example, approximately 1 to 5 milligrams per day of a honokiol-type compound can reduce the volume of a solid tumor in mice. In particular, administration of approximately 3 milligrams daily of the honokiol-type compound reduces the solid tumor more than fifty percent, as discussed in Bai et al., Isolation, characterization, and antitumor activity of a small molecular weight compound from Magnolia grandiflora. These results can be used to predict an approximate amount of the honokiol-type compound to be administered to a human.

The approximation includes host factors such as surface area, weight, metabolism, tissue distribution, absorption rate, and excretion rate, for example. Therefore, approximately 0.5 to 7 grams per day of the honokiol-type compound and/or the magnolol-type compound should produce similar results in humans. In particular, approximately 1 to 4 grams per day of the honokiol-type compound and/or the magnolol-type compound can be administered to humans to produce similar results. As stated above, a therapeutically effective dose level will depend on many factors, as described above. In addition, it is well within the skill of the art to start doses of the composition at relatively low levels, and increase the dosage until the desired effect is achieved.

Compositions of the present invention may be used in combination with other compositions and/or procedures for the treatment of the conditions described above. For example, a tumor may be treated conventionally with surgery, radiation, or chemotherapy combined with one or more compositions of the present invention and then one or more compositions of the present invention may be subsequently administered to the patient to extend the dormancy of micrometastases and to stabilize, inhibit, or reduce the growth of any residual primary tumor.

In particular, compositions of the invention may also be combined with other antiangiogenic agents to enhance their effectiveness, or combined with other antiangiogenic agents and administered together with other cytotoxic agents. In particular, when used in the treatment of solid tumors, compositions of the invention may be administered with IL-12, retinoids, interferons, angiostatin, endostatin, thalidomide, thrombospondin-1, thrombospondin-2, captopryl, anti-neoplastic agents such as alpha interferon, COMP (cyclophosphamide, vincristine, methotrexate and prednisone), etoposide, mBACOD (methortrexate, bleomycin, doxorubicin, cyclophosphamide, vincristine and dexamethasone), PRO-MACE/MOPP (prednisone, methotrexate (w/leucovin rescue), doxorubicin, cyclophosphamide, taxol, etoposide/mechlorethamine, vincristine, prednisone and procarbazine), vincristine, vinblastine, angioinhibins, TNP-470, pentosan polysulfate, platelet factor 4, angiostatin, LM-609, SU-101, CM-101, Techgalan, thalidomide, SP-PG and the like, as well as with radiation.

Compositions of the present invention may be used with a sustained-release matrix. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

As indicated above, compositions of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically-acceptable and metabolizable lipid capable of forming liposomes can be used. The liposome can contain, in addition to one or more compositions of the present invention, stabilizers, preservatives, excipients, and the like. The preferred lipids are the phospholipids and the phosphatidyl cholines (lecithins), both natural and synthetic. Methods to form liposomes are known in the art.

It should be emphasized that the above-described embodiments of the present invention are merely possible examples of implementations, and are set forth only for a clear understanding of the principles of the invention. Many variations and modifications may be made to the above-described embodiments of the invention without departing substantially from the spirit and principles of the invention. All such modifications and variations are intended to be included herein within the scope of this disclosure and the present invention and protected by the following claims.

Therefore, having thus described the invention, at least the following is claimed:

1. A method of treating a condition comprising administering to a host in need of treatment an effective amount of at least one honokiol-type compound.

2. The method of claim 1, wherein the condition is characterized by angiogenesis.

3. The method of claim 1, wherein the condition is characterized by tumorogenesis.

4. The method of claim 1, wherein the condition is characterized by a neoplastic condition.

5. The method of claim 1, wherein the condition is cancer.

6. The method of claim 1, wherein the condition is a skin disorder.

7. The method of claim 1, wherein the at least one honokiol-type compound has the formula of structure A1:

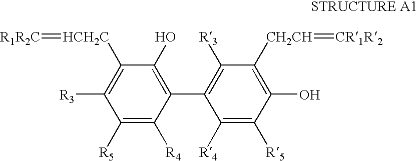

8. The method of claim 7, wherein at least one of the functional groups R₁, R₂, R₃, R₄, R₅, R′₁, R′₂, R′₃, R′₄, and R′₅ of structure A1 is chosen from hydrogen, hydroxyl groups, amides, amines, hydrocarbons, halogenated hydrocarbons, cyclic hydrocarbons, cyclic heterocarbons, halogenated cyclic heterocarbons, benzyl compounds, halogenated benzyl compounds, organo selenium compounds, sulfide compounds, cabonyl compounds, thiol compounds, ether compounds, dinitrogen ring compounds, thiophene compounds, pyridine compounds, pyrrole compounds, imidazole compounds, and pyrimidine compounds.

9. The method of claim 1, wherein the at least one honokiol-type compound has the formula of structure A2:

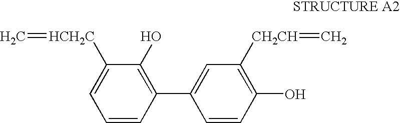

10. The method of claim 1, wherein the at least one honokiol-type compound is chosen from honokiol-type compound analogues, honokiol-type compound homologues, honokiol-type compound isomers, and honokiol-type compound derivatives.

11. The method of claim 1, wherein the at least one honokiol-type compound includes pharmaceutically acceptable salts of the honokiol-type compounds.

12. The method of claim 1, wherein the at least one honokiol-type compound includes pharmaceutically acceptable prodrugs of the honokiol-type compounds.

13. The method of claim 1, further comprising administering to the host in need of treatment an effective amount of at least one magnolol-type compound.

14. A method of treating a condition comprising administering to a host in need of treatment an effective amount of at least one magnolol-type compound.

15. The method of claim 14, wherein the condition is characterized by angiogenesis.

16. The method of claim 14, wherein the condition is characterized by tumorogenesis.

17. The method of claim 14, wherein the condition is characterized by a neoplastic condition.

18. The method of claim 14, wherein the condition is cancer.

19. The method of claim 14, wherein the condition is a skin disorder.

20. The method of claim 14, wherein the at least one magnolol-type compound has the formula of stricture B1:

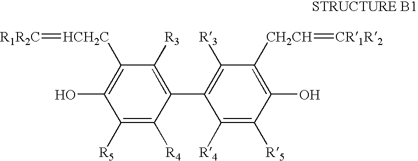

21. The method of claim 20, wherein at least one of the functional groups R₁, R₂, R₃, R₄, R₅, R′₁, R′₂, R′₃, R′₄, and R′₅ of structure B1 is chosen from hydrogen, hydroxyl groups, amides, amines, hydrocarbons, halogenated hydrocarbons, cyclic hydrocarbons, cyclic heterocarbons, halogenated cyclic heterocarbons, benzyl compounds, halogenated benzyl compounds, organo selenium compounds, sulfide compounds, cabonyl compounds, thiol compounds, ether compounds, dinitrogen ring compounds, thiophene compounds, pyridine compounds, pyrrole compounds, imidazole compounds, and pyrimidine compounds.

22. The method of claim 14, wherein the at least one magnolol-type compound has the formula of structure B2:

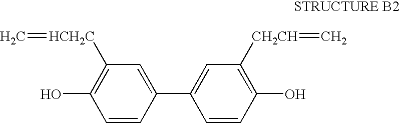

23. The method of claim 14, wherein the at least one magnolol-type compound is chosen from magnolol-type compound analogues, magnolol-type compound homologues, magnolol-type compound isomers, and magnolol-type compound derivatives.

24. The method of claim 14, wherein the at least one magnolol-type compound includes pharmaceutically acceptable salts of the magnolol-type compounds.

25. The method of claim 14, wherein the at least one magnolol-type compound includes pharmaceutically acceptable prodrugs of the magnolol-type compounds.

26. The method of claim 14, further comprising administering to the host in need of treatment an effective amount of at least one honokiol-type compound.

27. A chemopreventative method of treating a condition comprising administering to a host in need of treatment an effective amount of at least one honokiol-type compound.

28. The chemopreventative method of claim 27, wherein the at least one honokiol-type has the formula of structure A1.

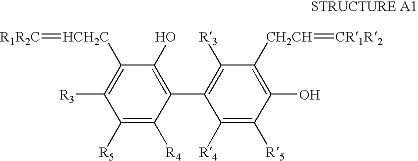

29. The chemopreventative method of claim 27, further comprising administering to the host in need of treatment an effective amount of at least one magnolol-type compound.

30. The chemopreventative method of claim 27, wherein the at least one magnolol-type compound has the formula of structure B 1:

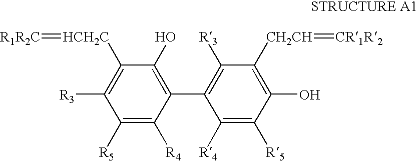

31. A pharmaceutical composition comprising at least one magnolol-type compound in combination with a pharmaceutically acceptable carrier, wherein the at least one magnolol-type compound is present in a dosage level effective to treat a condition.

32. The pharmaceutical composition of claim 31, wherein the at least one magnolol-type compound has the formula of structure B 1:

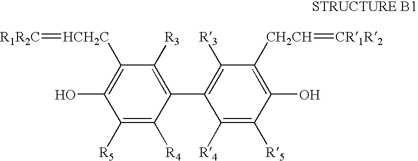

33. The pharmaceutical composition of claim 31, further comprising at least one honokiol-type compound present in a dosage level effective to treat the condition.

34. The pharmaceutical composition of claim 31, wherein the at least one honokiol-type compound has the formula of structure A1: [chemical structure]
